(19) United States
(12) Patent Application Publication
(10) Pub. No.: US 2017/0175070 A1
(43) Pub. Date: Jun. 22, 2017

(54) In Situ Biomass Generator with Automated Disinfection

(71) Applicant: NCH Corporation, Irving, TX (US)

(72) Inventors: Scott Boyette, Irving, TX (US); William P. Boesch, Dallas, TX (US); Alexander C. Erdman, Irving, TX (US); Mike Rushing, Denton, TX (US); Judith G. Pruitt, Mesquite, TX (US); Daniel Aberle, Irving, TX (US); Andrew McKinnon, Irving, TX (US)

(21) Appl. No.: 15/383,808

(22) Filed: Dec. 19, 2016

Related U.S. Application Data
(60) Provisional application No. 62/268,676, filed on Dec. 17, 2015.

(51) Int. Cl. C12M 1/12; C12M 1/36; C12M 1/34; C12M 1/00

(52) U.S. Cl. CPC C12M 37/00; C12M 23/40; C12M 29/00; C12M 41/48; C12M 41/12; C12M 29/20

(57) ABSTRACT

A self-contained biomass generator configured for use in growing and discharging bacteria at or near an intended use site and having a housing containing a bacteria growth chamber; water and air inlets; receptacles and solenoid pumps for liquids containing bacteria spores, nutrient, defoamer and disinfectant; a recirculation pump; air pump; water heater; manifold; a dosing pump configured to dispense controlled amounts of bacteria; and a programmable electronic controller configured to operate the device through a plurality of bacteria growth cycles and to disinfect the wetted internal surfaces between growth cycles without requiring service or operator intervention. A process for using the biomass generator is also disclosed.

### BACKGROUND OF THE INVENTION

1. Field of the Invention

This invention relates to biomass generators that grow and discharge bacteria for beneficial end uses such as remediation of contaminates, control of pests, production of micro-organisms, and reduction of odors. More particularly, this invention relates to an in situ bacteria production system and apparatus configured to grow utility populations of probiotic bacteria suitable for use with animals, in animal feeds, and in water supplies to promote overall health and metabolic efficiency. As used throughout this disclosure, “in situ” refers to biomass generators in which utility populations of useful bacteria that are sufficient for a particular application are grown at or near the intended use site. The invention also relates to an in situ bacteria production system and apparatus that is configured to filter air introduced into the apparatus and to automatically sanitize or disinfect the apparatus between bacteria growth cycles. Inlet air filtration and automated disinfection are disclosed in relation to the subject apparatus to reduce the likelihood of contamination by other undesirable bacterial species and aid in the removal of films or solids remaining in the mixing chambers or flow lines following harvesting of the beneficial bacteria. Use of the system and apparatus disclosed here will desirably automatically disinfect all wetted surfaces between successive bacteria growing cycles within a normal service interval. This will in turn improve performance of the apparatus while avoiding the need for intervention by a service technician during a 30-60 day service interval and in some circumstances will enable the service interval to be extended for longer periods, such as up to about 90 days or more, where sufficient volumes of the needed materials are provided.

2. Description of Related Art

Systems and devices useful for growing and harvesting bacteria for useful applications are well known. Such systems are often designed and implemented on an industrial scale, with a large footprint, complex temperature, pressure and mixing systems requiring substantial capital investment, significant energy demands, offsite utilities, and highly trained operators and maintenance personnel.

More recently, smaller scale biomass generators have been developed and disclosed that can reliably produce aqueous suspensions of bacteria. Such devices are disclosed, for example, in U.S. Pat. Nos. 6,335,191; 7,081,361; 7,635,587; 8,093,040; and 8,551,762, but typically lack clean in place (CIP) functionality and require some disassembly in order to clean and disinfect the interior surfaces of component parts such as vessels, flow lines, valves and pumps.

In situ biomass generators having more automated and effective disinfection systems and methods are therefore needed to better protect against the unintended production of harmful organisms inside the mixing chambers and fluid flow lines of the apparatus that can sometimes lead to plugged flow lines or contaminated product.

### SUMMARY OF THE INVENTION

The in situ biomass generators disclosed here are desirably configured to include an automated disinfection capability to facilitate automatic cleaning of the wetted portions of such devices according to a regular and reliable disinfection procedure that can be effectuated after each growing cycle without the assistance or intervention of a service technician. When a regularly scheduled service call does occur, the service technician can typically perform a visual inspection and change out the containers of liquid bacteria, nutrient and disinfectant that are provided for use in the apparatus without having to disassemble and separately clean each component part and flow line. This in turn produces better product and allows longer service intervals, with lower associated operating costs.

One embodiment of the subject invention desirably includes a housing with a latchable door that is suitable for mounting on a wall or other support surface proximal to the use environment. The housing desirably contains a bacteria growth chamber; an inlet manifold in which a bacteria growth medium comprising water, a suspension of starter bacteria and a liquid nutrient broth can be pre-mixed and introduced into the growth chamber; a recirculation pump configured to receive the bacteria growth medium from an outlet port in the bottom of the growth chamber and discharge it into an outlet manifold in which the growth medium can be heated and aerated before being returned to the bacteria growth chamber. Inlet air entering the apparatus is desirably filtered prior to contacting the bacteria-containing solutions to reduce the likelihood of contamination by other air-borne bacteria. The flow of water into the growth chamber is desirably at line pressure and is controlled by an inlet valve and flow meter also disposed within the housing. At the end of each bacteria growth cycle, the resultant bacteria can be harvested by redirecting the outlet flow from the recirculation pump and into a drain for immediate use or into another accumulator or storage container for subsequent use or dilution.

A plurality of containers separately holding liquid suspensions of starter bacteria and the liquid nutrient broth can also be disposed inside the same housing as the bacteria growth chamber, but are desirably positioned inside a second enclosure that is located nearby and connected by flexible flow lines to a plurality of peristaltic pumps disposed inside the first housing. An electronic control panel is desirably provided inside the first housing and desirably comprises a timer and temperature and flow controllers that are programmable and are adjustable by a service technician during initial set-up and afterward if needed to adjust the pumps, valves, fluid flows and fluid temperature as are suitable for continuous operation of the system throughout each service interval. An external power source is desirably provided and wired into the housing to provide electricity to the control panel.

In accordance with the present invention, a container comprising liquid sanitizer or disinfectant (referred to below as “disinfectant”) is also provided inside the housing or the other enclosure for use in washing down and treating the wetted surfaces of the apparatus following discharge of the harvested bacteria at the end of each growth cycle during a service interval. Another peristaltic pump is also desirably provided inside the housing to receive liquid disinfectant from the disinfectant storage container and discharge it into the inlet manifold to be mixed with water and then recirculated through the growth chamber and ancillary equipment by the recirculation pump to achieve the desired cleaning prior to commencing the next bacteria growth cycle. Satisfactory disinfectants suitable for use in the system and apparatus of the invention can include, for example and without limitation, aqueous solutions of bleach, hydrogen peroxide, and quaternary ammonium compounds.

The system and apparatus of the invention can be characterized as “self-cleaning” in that the inclusion of a sanitizer or disinfectant inside the system and the capability of the apparatus to automatically circulate the disinfectant throughout the wetted surfaces of the apparatus at the end of each bacteria growth cycle reduces the likelihood of film or solids accumulation inside the apparatus and reduces the need for having a technician disassemble and clean individual parts. The subject system and apparatus are also characterized by a minimal footprint where desired, but are scalable for use environments requiring larger volumes of bacteria growth medium. The system and apparatus embody a unique flow design, precision liquid controls, and low energy requirements, can be operated by solar, battery or direct electrical power, and can be configured or adapted to operate by batch, batch continuous or continuous processing.

According to another embodiment of the invention, rather than providing and circulating a liquid disinfectant to disinfect the wetted surfaces, the apparatus is configured to automatically heat sterilize the recirculating bacteria growth medium prior to starting the bacteria growing cycle by heating and recirculating the liquid at a temperature of about 65° C. for one hour, and then allowing it to cool to about 35° C. before beginning timing of the bacteria growing cycle. Alternatively, the apparatus is configured to automatically heat the recirculating purge water remaining in the apparatus following harvesting of the bacteria growth medium to a temperature of about 65° C. for one hour, after which the recirculating water is allowed to cool to a lower temperature before introducing additional bacteria-containing liquid and liquid nutrient to commence another bacteria growth cycle. As used in this embodiment, “heat sterilize” means to raise the temperature of the recirculating liquid to a temperature and for a period of time that are sufficient to purge the system of harmful bacteria without also eliminating the desirable bacteria. Heating the recirculating liquid to a temperature of about 65° C. for about one hour is believed to be sufficient for achieving this purpose. In this embodiment, an external heater can be provided through which the bacteria growth medium or water can be recirculated to reach and maintain the desired temperature.

Suitable applications for use of the bacteria produced in the system and apparatus of the invention can include, for example and without limitation, food manufacturing and restaurant operations; farming and livestock operations; landscaping operations; sewage and water treatment operations, and the like.

### BRIEF DESCRIPTION OF THE DRAWINGS

The system and apparatus of the invention are further described and explained in relation to the accompanying drawing figures wherein:

FIG. 1 is a simplified right front perspective view of the principal components of one embodiment of the apparatus of the invention, with the front cover of the housing and the fluid flow lines removed in order to simplify the illustration for clarity;

FIG. 2 is a front elevation view of the apparatus of FIG. 1;

FIG. 3 is a further-modified version of the apparatus of FIG. 1 that incorporates some of the fluid flow lines, adds dosing pump 146 and does not include the optional heater shown in FIG. 1;

FIG. 4 is an enlarged detail view taken from FIG. 3;

FIG. 5 is a further enlarged front elevation view of recirculating pump 20 and recirculation manifold 22 of FIG. 4;

FIG. 6 is an enlarged front elevation view of the apparatus of FIG. 3;

FIG. 7 is a front elevation view of a second enclosure comprising a plurality of containers separately holding liquid suspensions of starter bacteria or bacteria spores, nutrient broth, and disinfectant;

FIG. 8 is a right front perspective view of the principal components of another embodiment of an in situ biomass generator of the invention, with a front door of the apparatus being shown in an open position;

FIG. 9 is a front elevation view of the apparatus of FIG. 8, with a portion of the open door broken away;

FIG. 10 is an enlarged detail view taken from FIG. 9;

FIG. 11 is a further enlarged detail view of manifold 225 of FIG. 10;

FIG. 12 is a left front perspective view of the apparatus of FIG. 9, also having a portion of the open door broken away;

FIG. 13 is a diagrammatical process flow diagram showing for illustrative purposes the principal component parts of the apparatus of FIGS. 8-12 and the fluid flow paths through them; and

FIG. 14 is diagrammatic flow diagram showing for illustrative purposes a representative operating cycle for the apparatus of FIGS. 8-12.

### DETAILED DESCRIPTION OF EMBODIMENTS

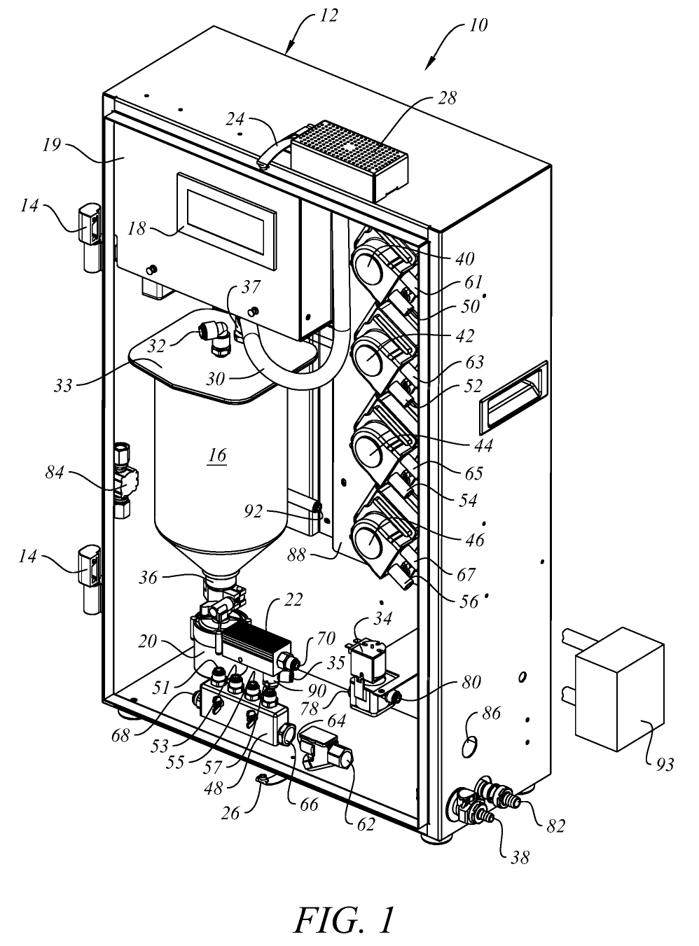

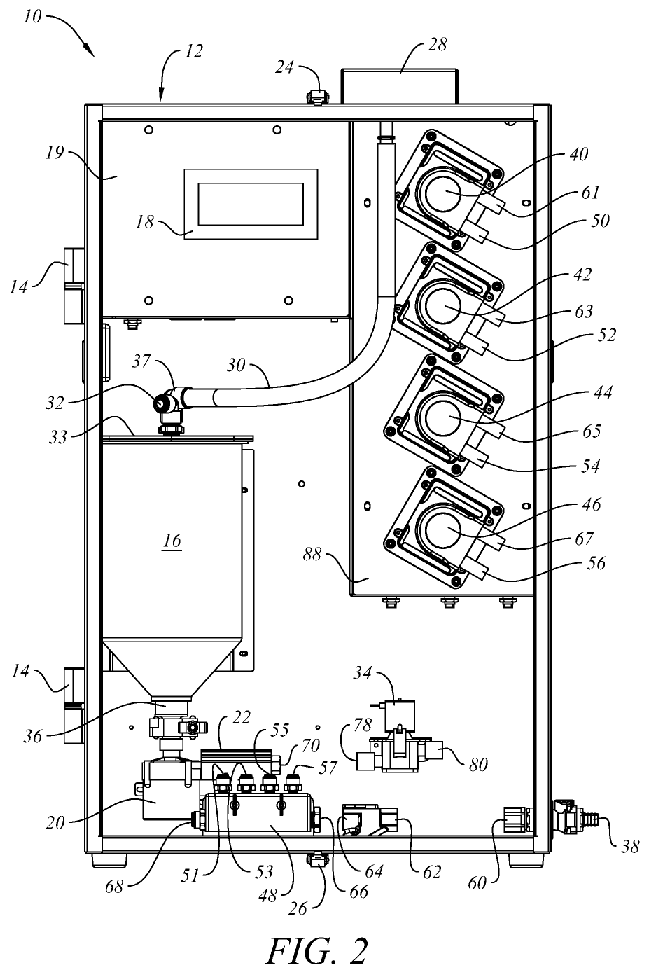

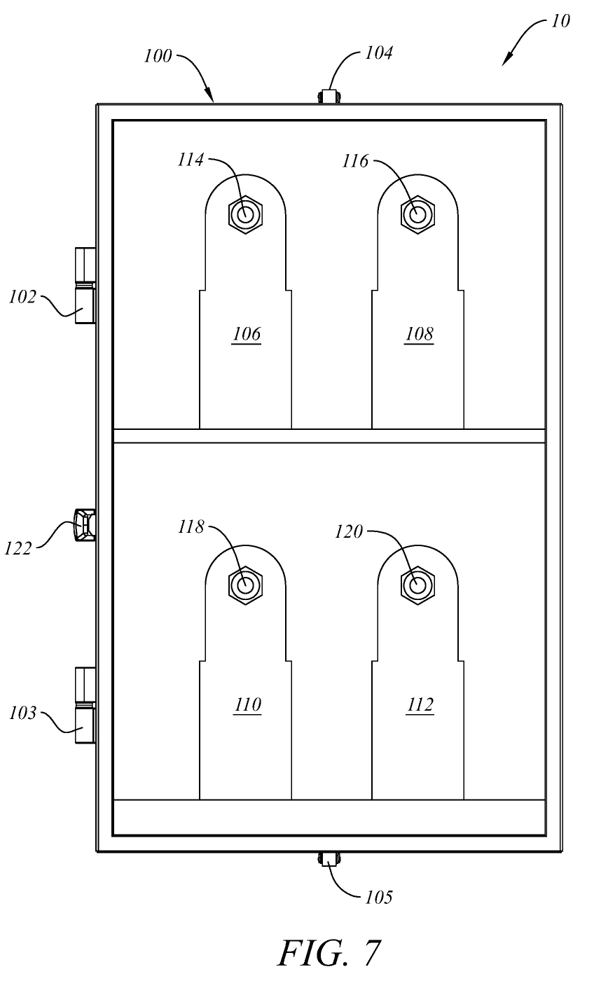

Referring to FIGS. 1-2, one embodiment of a system comprising in situ biomass generator 10 (hereafter referred to as “biomass generator 10” for convenience) of the invention is disclosed that further comprises housing 12 attachable to a wall (not shown) or other supporting surface. Housing 12 desirably has top, bottom and side walls, and a front cover (not shown) that is attached by hinges 14 and can be latched, for example, by top and bottom latches 24, 26, respectively, and preferably locked by a conventional rotatable key lock (not shown) mounted on the cover. Referring to FIG. 7, a second lockable enclosure 100 is also desirably provided as part of biomass generator 10 for storage of a plurality of media containers such as containers 106, 108, 110, and 112 in proximity to housing 12. Enclosure 100 desirably includes a lockable cover (not shown) with hinges 102, 103 and latches 104, 105, together with a plurality of outlet fittings 122 sufficient to accommodate fluid flows from the media containers in enclosure 100 to their respective pumps in housing 12.

Each of containers 106, 108, 110 and 112 desirably contains a liquid comprising starter bacteria, nutrient broth, defoamer or disinfectant and is connected by a flexible flow line to one of pumps 40, 42, 44, 46, respectively disposed inside housing 12 (FIG. 1). It will be appreciated that the number of media containers and the contained volume of each container can vary as needed to provide sufficient raw materials for sustaining bacteria growth during multiple growth cycles and for automatically disinfecting wetted surfaces of the apparatus at the end of each growth cycle. The subject invention will desirably function throughout a predetermined service interval—such as, for example, from about 30 to about 90 days—without requiring personal intervention or servicing by a technician. Fluid flow lines and electrical lines between constituent elements of the apparatus are not shown in FIGS. 1-2, 7 to simplify the drawings for illustrative purposes, but their placement will be understood by those of skill in the art upon reading this disclosure in relation to the other figures of the accompanying drawings. Fluid flow lines are desirably made of flexible, transparent or translucent polymeric materials that are non-reactive with the fluids passing through them, but any other similarly effective conduit can likewise be used within the scope of the invention.

Referring to FIGS. 1-2, 7, biomass generator 10 is desirably configured for attachment to a conventional water supply line by fittings 38 and 60 that cooperatively define an inlet port through the wall of housing 12. Incoming water desirably flows from inlet fitting 60 into normally closed solenoid valve 62 into inlet 66 of inlet manifold 48. Inside inlet manifold 48, the water is mixed with liquid starter bacteria and nutrient broth, each being supplied from at least one of media containers 106, 108, 110, 112 through an associated positive displacement pump, such as one of peristaltic pumps 40, 42, 44, 46. As an example, a liquid suspension of starter bacteria is drawn from container 106 through a flow line connected to outlet fitting 114 of container 106 and to inlet fitting 61 of pump 40. The fluid flow line from container 106 to pump 40 desirably exits enclosure 100 through fittings 122 and enters housing 12 through aperture 86 (FIG. 1). A liquid suspension (“broth”) of nutrient material is similarly drawn from container 108 through a flow line connected between outlet fitting 116 and inlet fitting 63 of pump 42. At least one of the other media containers 110, 112 and pumps 44, 46, can be used to store a disinfectant such as, for example, liquid bleach for use at the end of each bacteria growth cycle. The volumes of starter bacteria, nutrient broth and disinfectant needed for a single growth cycle can be determined according to the volume of the bacteria growth chamber 16 being used. The number and volume of media containers needed to store those respective materials can then be calculated by multiplying the volume required for each growth cycle by the number of growth cycles during a desired service interval. It should also be understood that more than one medium container from enclosure 100 can be connected to a single pump inlet.

In the embodiment described in relation to FIGS. 1-2, 7, starter bacteria from outlet 50 of pump 40 is introduced into inlet manifold 48 through inlet port 51, where it is premixed with water introduced through inlet 66 and with nutrient broth supplied to inlet manifold 48 through inlet port 53 from outlet port 52 of pump 42 to form a bacteria growth medium. The growth medium exits through outlet 68 of inlet manifold 48 and flows upwardly through flow meter 84 into inlet port 32 through cover 33 of bacteria growth chamber 16. Although satisfactory results are achieved using a bacteria growth chamber 16 having a usable internal volume of about three liters, it will be appreciated that growth chambers having greater or lesser volumes can also be used within the scope of the invention. Vent line 30 is desirably provided to vent the inside of growth chamber 16 to atmosphere between vent port 37 of cover 33 and protective lid 28 on top of housing 12. Protective lid 28 shields the inlet/outlet air fittings (not visible) from being damaged from the outside, and is desirably perforated to act as an insect screen and to otherwise prevent small objects from entering growth chamber 16 through vent port 37. The air inlet line (discussed below) is routed under panel 88 to which peristaltic pumps 40, 42, 44 and 46 are mounted.

Once a predetermined volume of bacteria growth medium as determined by flow meter 84 (visible in FIG. 1) is charged into growth chamber 16, inlet valve 62 is closed and recirculating pump 20 disposed beneath outlet port 36 of growth chamber 16 is activated, causing the bacteria growth medium discharged from recirculating pump 20 to enter recirculation manifold 22 and then exit recirculation manifold 22 through outlet port 70. Recirculation manifold 22 is desirably an aluminum block having a resistance heating element adhered directly to the manifold to heat the growth medium to an acceptable growth temperature as determined according to the bacteria type and other related factors. While passing through recirculation manifold 22, the fluid temperature is desirably sensed by RTD temperature probe 144 (FIG. 5) or equivalent and sent back to the electronic controller mounted behind panel 19 of housing 12, which then controls operation of the resistance heating element. A flow control valve 90 (FIG. 1) is desirably configured to inject a flow of pressurized air received through inlet port 35 directly into the recirculating growth medium passing through recirculation manifold 22. The air is injected into the growth medium downstream of the recirculating pump to avoid cavitation, but sufficiently in advance of re-entering growth chamber 16 that the air bubbles are in contact with the growth medium in the recirculation loop for a period of time sufficient to oxygenate the temperature-controlled suspension of growing bacteria to promote additional growth inside growth chamber 16.

Upon exiting outlet port 70 of recirculation manifold 22, the growth medium desirably enters solenoid valve 34 through inlet port 78. Solenoid valve 34 is desirably normally open to a recirculation line through which the flow of growth medium re-enters growth chamber 16 tangentially through recirculation inlet line 92, but can be selectively reconfigured by electronic controller 18 to harvest the bacteria growth medium at the end of the bacteria growing cycle. This is done by discharging the flow of growth medium received from recirculation manifold 22 through outlet 80 and outlet port 82 in housing 12 into an external drain line (not shown). Solenoid valve 34 is desirably reconfigured automatically by a signal received from electronic controller 18 when the internal timer has reached the predetermined set point for harvesting the bacteria.

As soon as the growth medium is harvested following a growing cycle, solenoid valve 34 is returned to a recirculation mode and solenoid valve 62 desirably reopens to restart the flow of pressurized water into inlet manifold 48. At or near the same time, another peristaltic pump mounted on panel 88 is activated to draw disinfectant from another of the media containers disposed inside second enclosure 100 into inlet manifold 48 to be mixed with the incoming pressurized water before flowing through flow meter 84 (FIG. 1) into growth chamber 16. Once a desired volume of mixed water and disinfectant have been charged to growth chamber 16, recirculation pump 20 is reactivated to begin circulating the water and disinfectant to purge any remaining contaminants or undesirable blockages present in the system.

Representative bacteria spores satisfactory for growing in the apparatus of the invention can include, for example and without limitation:
B. amyloliquefaciens
B. clausii
B. circulars
B. coagulans
B. firmus
B. lactis
B. laterosporus
B. laevolacticus
B. lentus
B. licheniformis
B. megaterium
B. mucilaginosus
B. mycoides
B. polymyxa
B. polyfermenticus
B. pumilus
B. simplex
B. sphaericus
B. subtilis
B. subtilis natto
   Representative nutrient materials satisfactory for use in growing such bacteria inside biomass generator 10 can include, for example, nutrient broths comprising aqueous suspensions of organic materials, such as but not limited to, sugars, carbohydrates, proteins, fats; and buffers, pH adjusters, preservatives, spore activator, or any beneficial compounds. In general, the bacteria selected for use in biomass generator 10 are those known to be effective for the intended end-use, and the nutrient broth is selected to include components known to be useful and effective for growing the selected bacteria. A suitable defoamer is sunflower oil. Suitable sanitizers or disinfectants for use in the system and apparatus of the invention can include, for example and without limitation, solutions of bleach, hydrogen peroxide and quaternary ammonium compounds. Use of the subject apparatus is further described below in relation to the following Example 1:

### Example 1

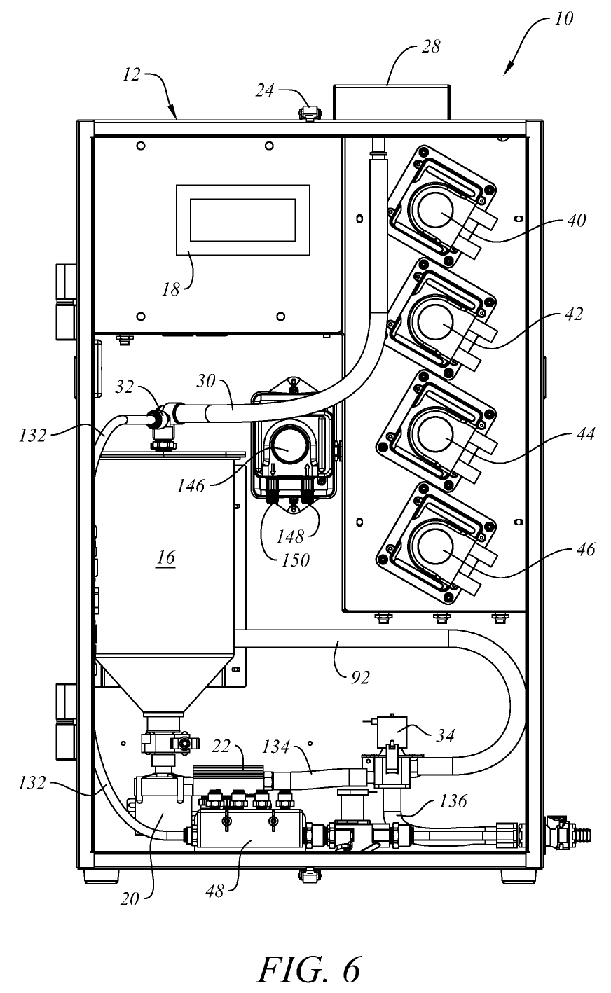

Using the system and apparatus of the invention as disclosed here in one satisfactory mode of operation, a liquid disinfection agent or mixture of disinfection agents is automatically dispensed into the biomass generator mixing manifold, flushed into the growth chamber with 80% of full water charge, and allowed to circulate for a pre-determined time. The circulation is then interrupted; the remaining fermentation components are introduced into the mixing manifold, and the remaining 20% of full water charge is used to flush the bacteria growth components through the system and in the mixing vessel. The mixing vessel circulation is then restarted and normal the normal bacteria growth (fermentation) process continues. At the completion of the biomass fermentation process, the fermentation liquid is dispensed. A rinse cycle process is initiated by injecting a small amount of disinfecting agent into the mixing manifold which is flushed into the system and mixing vessel with a 30% of full water charge, allowed to circulate for a minimum of 1 minute, and then discharged to drain. Although not shown in FIGS. 1 and 2, drain line 136 is visible beneath solenoid valve 34 in the embodiment shown in FIGS. 4 and 6. This process is repeated two more times. At the end of the third rinse, the fermentation process is re-initiated and another bacteria growth cycle begins.

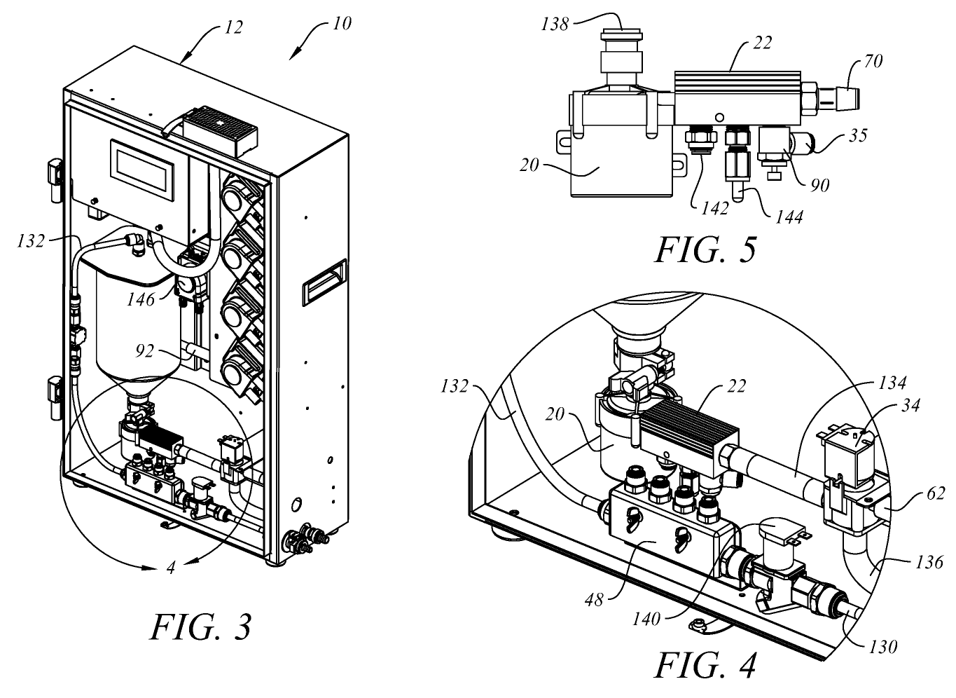

Referring to FIGS. 3-6, biomass generator 10 is further described and modified in accordance with another embodiment of the invention. Looking first at FIGS. 3-4, water at line pressure enters housing 12 through line 130 and solenoid valve 140, and enters inlet manifold 48. A conventional float switch (not shown) is desirably also provided to sense any accumulated liquid in the bottom of housing 12 and shut down operation of biomass generator 10. Referring to FIGS. 1 and 6, water line 132 exiting inlet manifold 48 communicates with bacteria growth chamber 16 through flow meter 84 (identified in FIG. 1). Referring to FIGS. 4-6, recirculation pump 20 disposed beneath bacteria growth chamber 16 discharges liquid received from bacteria growth chamber 16 into recirculation manifold 22. While inside recirculation manifold 22, the liquid can be heated by an adhesive resistance heater attached to recirculation manifold 22 or by another similarly effective heater. For example, optional heater 93 (shown in FIG. 1) can be connected to flow line 132 between flow meter 84 and inlet port 32 in lid 33 of bacteria growth chamber 16.

Referring to FIGS. 1, 4-6, air control valve 90 as previously described in relation to FIG. 1 is disposed beneath recirculation manifold 22 for use in injecting pressurized air received through port 86 of housing 12 into liquid being recirculated from recirculation manifold 22 back to bacteria growth chamber 16 through flow line 134, solenoid valve 34 and recirculation inlet line 92. At the conclusion of each bacteria growth cycle, solenoid valve 34 can be repositioned to direct the recirculated fluid received from recirculation manifold 22 into drain line 136 that discharges the bacteria-containing liquid through outlet port 82 (FIG. 1). FIGS. 3 and 6 also depict the addition of dosing pump 146 with inlet 148 and outlet 150 as an optional modification to biomass generator 10 that can be utilized to carefully control the discharge of comparatively smaller amounts of bacteria-containing growth media from biomass generator 200 into another vessel or conduit. According to one method or process of the invention, dosing pump 146 is activated during the last two hours of each bacteria growth cycle to dispense controlled amounts of bacteria-containing growth media during that period. For example, and without limitation, dosing pump 146 can receive bacteria growth medium containing a probiotic bacteria through inlet port 148 from outlet port 142 (FIG. 5) on recirculation manifold 22 and dispense relatively small quantities metered through outlet port 150 and into a discharge line (not shown) through a wall of housing 12 into another flow line external to biomass generator 10 for an end use application. According to one satisfactory application of biomass generator 10, the bacteria growth medium comprising probiotic bacteria is discharged into a pressurized water line supplying drinking water to animals or supplying water for use in other animal husbandry or agricultural applications.

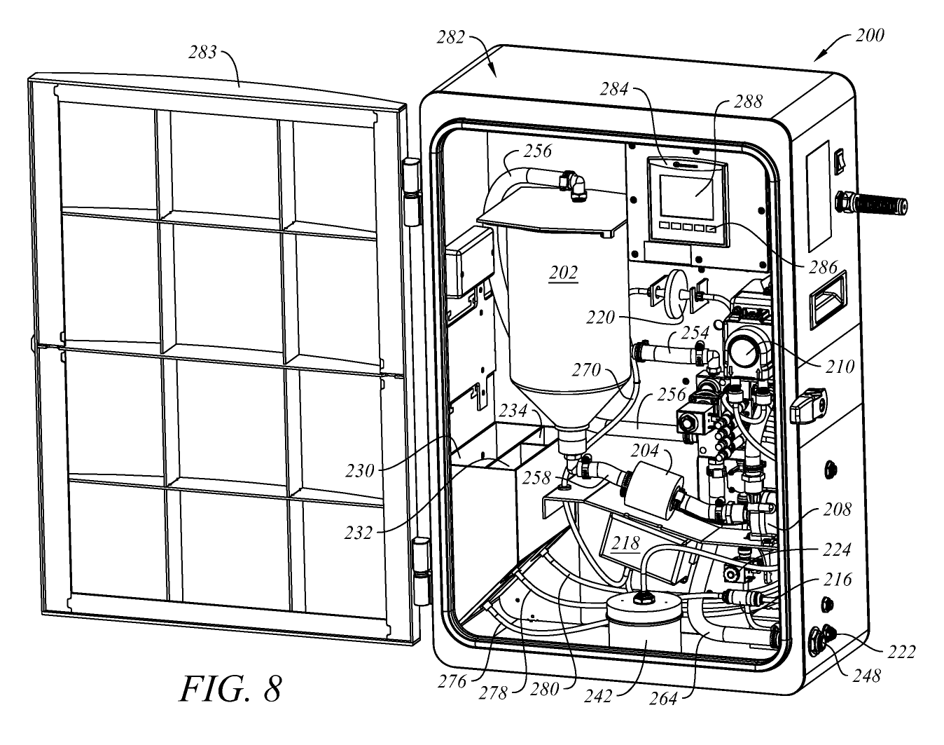

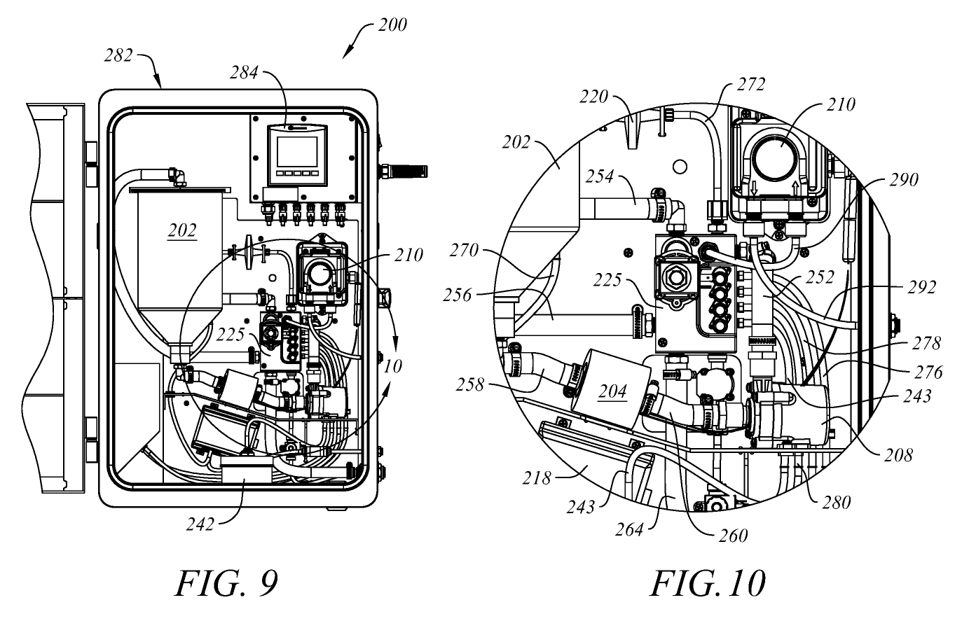

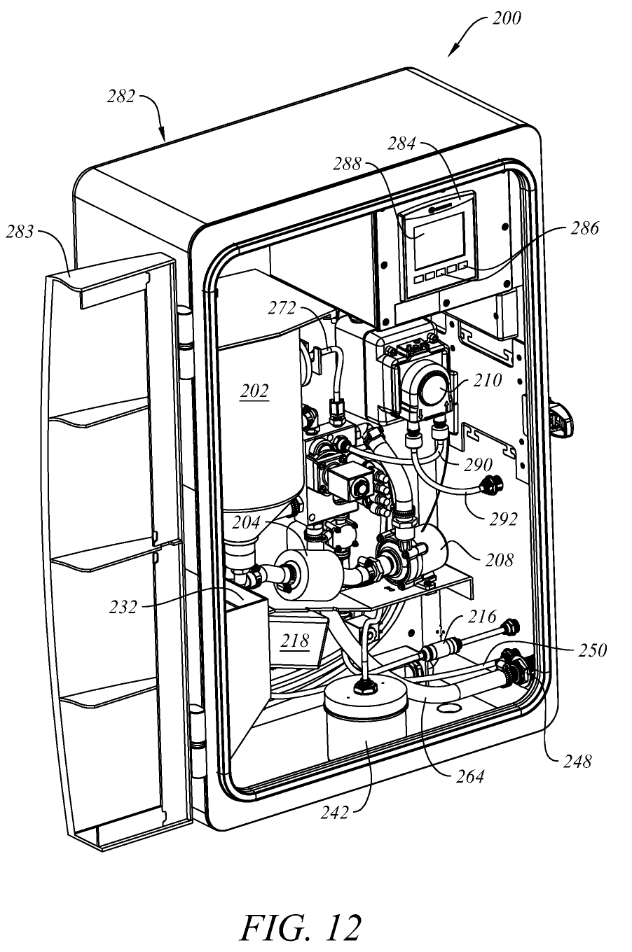

Referring to FIGS. 8-13, biomass generator 200, another embodiment of the in situ biomass generator of the invention, is disclosed that is satisfactorily disposed inside housing 282 having a hinged, preferably lockable door 283. Door 283 is desirably engageable with the seal visible around the perimeter of the opening into housing 282 as shown in FIGS. 8-9, 11 Biomass generator 200 differs from the embodiments disclosed in relation to FIGS. 1-7 in several ways. Once such difference is that receptacles 230, 232, 234 can support collapsible bags containing liquid nutrient, bacteria and defoamer inside housing 280 rather than having those materials disposed outside the housing or in another housing as described above in relation to FIG. 7. Another difference is that disinfectant receptacle 242 is also provided inside housing 280 to support and draw liquid from a bottle of bleach or other similarly effective liquid disinfectant such as hydrogen peroxide or a quaternary ammonia compound. When configured in this way, biomass generator 200 is self-contained as to all raw material components (other than water or air) needed to grow and discharge utility populations of bacteria.

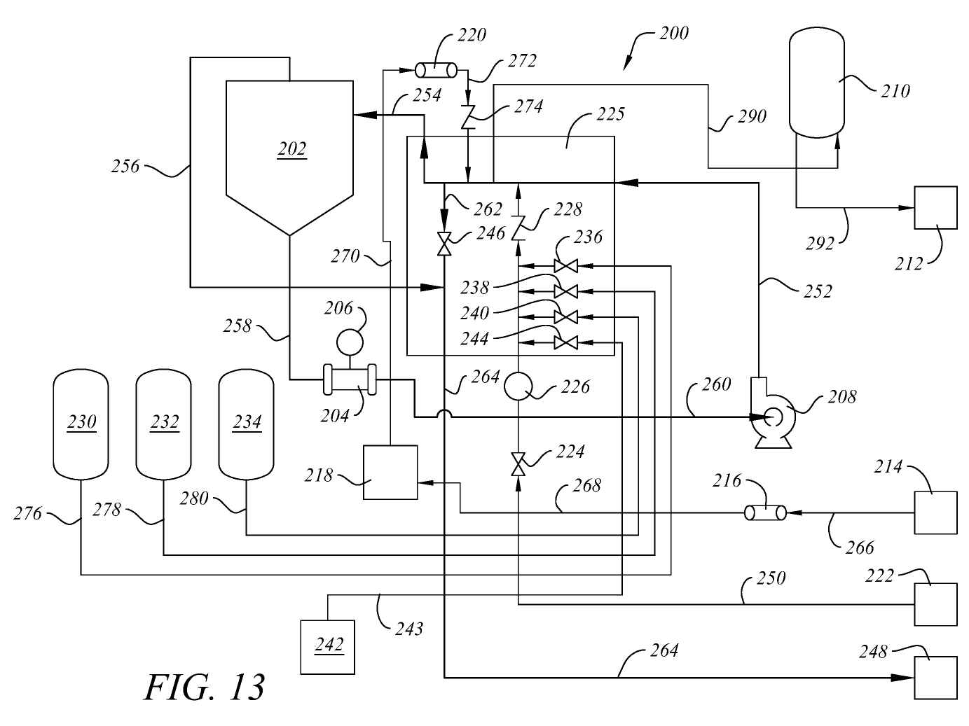

Another difference in the embodiment described in relation to FIGS. 8-13 is that the inlet and recirculation manifolds shown in the prior embodiment are combined into a single manifold 225. Another difference is that air pump 218 is provided to provide a source of pressurized, filtered air to bacteria growth chamber 202 through manifold 225. Another difference is that recirculation pump 208 is no longer disposed beneath bacteria growth chamber 202, and heater 204 is disposed between bacteria growth chamber 202 and recirculating pump 208. Another difference is that the function of the peristaltic pumps previously disclosed as elements 40, 42, 44, 46 of biomass generator 10 is now performed by solenoid pumps 236, 238, 240, 244 (FIGS. 11, 13) that are integrated into manifold 225 as best seen in FIG. 11. (For ease of reference, individual solenoid pumps 236, 238, 240, 244 are sometimes referred to collectively as “solenoid activated pumps 245” in this disclosure.)

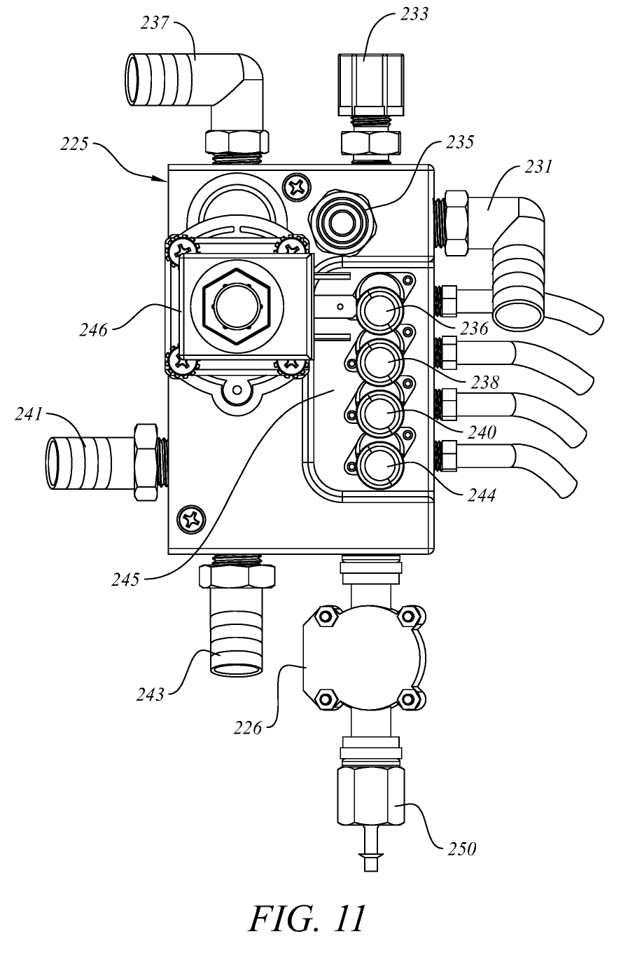

Referring to FIGS. 8-13, principal component parts of biomass generator 200 disposed inside housing 282 include bacteria growth chamber 202, which is constructed similarly to and in accordance with principles previously disclosed in prior art as noted in the Background section of this application, water heater 204, recirculation pump 208, dosing pump 210, inlet air filters 216, 220 (FIG. 8), manifold 225 (FIG. 10), flow meter 229 (FIG. 11), receptacles 230, 232, 234 and 242 for nutrient, bacteria spores, defoamer and disinfectant, respectively (FIG. 8), drain solenoid valve 239 (FIG. 11), solenoid activated pumps 245 for nutrient, bacteria spores, defoamer and disinfectant (FIG. 11), and an electronic controller 284 having control pad 286 and a digital display 288. Vent line 256 is desirably provided in the top of bacteria growth vessel to provide fluid communication with drain line 264 and drain outlet 248 in case of any pressure buildup inside bacteria growth chamber 202. Referring to FIG. 11, vent line 256 desirably enters drain line 264 downstream of drain solenoid valve 246 attached to manifold 225. A float switch (not visible in the views shown) similar in operation to float switch 140 discussed above is desirably provided with a sensor disposed near the bottom wall inside housing 282 that communicates with electronic controller 284 to shut down the operation if liquid accumulates inside the bottom of housing 280.

A universal power supply (not shown) is desirably provided that will work with 100-240 VAC, 50/60 Hz, MAINS power with short circuit, overload, voltage and temperature protections, and a 5 amp 240 VAC fuse on the MAINS side. The electronic components disposed inside housing 282 are all 24 VDC protected with self-resetting fuses. Biomass generator 200 is configured for use with a potable water supply at pressures ranging from about 2-80 psig (0.14 to 5.52 bar). A representative biomass generator 200 is configured to dispense about 3 liters of live active safe bacteria at the end of the growth cycle over a period of 2 hours. The bacteria can be dispensed through dosing pump 210, preferably a peristaltic pump, and outlet line 292 and outlet port 212 (FIG. 13). As disclosed here, biomass generator 200 is satisfactorily designed to operate in indoor or covered outdoor environments with temperatures ranging from about 37° F. (3° C.) to about 107° F. (42° C.), and is desirably not operated in direct sunlight. After each dosing cycle, biomass generator 200 rinses the wetted surfaces with a total of about 15 liters of combined water and disinfectant, and drains it from housing 282 through drain line 264 and outlet port 248 (FIGS. 8, 12, 13). The drain line is desirably open to the atmosphere and not pressurized, and biomass generator 200 is desirably installed in such manner that drain line 264 is always below drain port 248, with no intervening higher elevation.

Referring to FIGS. 8, 11-13, feed water is introduced into biomass generator 200 at line pressure through inlet port 222 and flow line 250, and passes through solenoid valve 224 and flow meter 226, and into manifold 225. Manifold 225 (best seen in FIG. 11) further comprises water inlet 250 through flow meter 226, air inlet 233, recirculation inlet 231, dosing outlet 235, solenoid activated pumps 245 (including nutrient solenoid pump 236, bacteria spore solenoid pump 238, defoamer solenoid pump 240 and disinfectant solenoid pump 244), drain solenoid valve 246, recirculation outlet 237, vent inlet 241 and drain outlet 243. Nutrient, bacteria spores, defoamer and disinfectant flow from receptacles 230, 232, 234 and 242, respectively, into manifold 225 through flow lines 276, 278, 280, 243, drawn by four independently controllable solenoid pumps 245 that discharge those components into the incoming water stream received into manifold 225 through flow line 250 (FIG. 13).

Ambient inlet air is received into housing 280 through inlet port 214, inlet air filter 216 and flow lines 266, 268 (FIG. 13), and enters the suction side of air pump 218. Air is discharged from air pump 218 at a pressure ranging from about 6 to about 10 psig and at a flow rate of about 2-6 scfh, and then flows at positive pressure through flow line 270, air filter 220, flow line 272 and check valve 274 into air inlet 233 of manifold 225 to be combined with the inlet water and other components entering manifold 225 through check valve 228 in flow line 250. The inlet air is desirably introduced into manifold 225 downstream of the point where those materials are discharged through check valve 228 in flow line 250. In one embodiment of the invention, the air flow is controlled by adjusting the speed of air pump 218. When drain solenoid valve 246 (FIGS. 11, 13) is closed, flow line 254 desirably discharges the combined materials tangentially into bacteria growth chamber 202, causing the liquids to swirl and continue mixing inside bacterial growth chamber 202. When biomass generator 200 is used to grow anaerobic bacteria, air pump 218 is not activated.

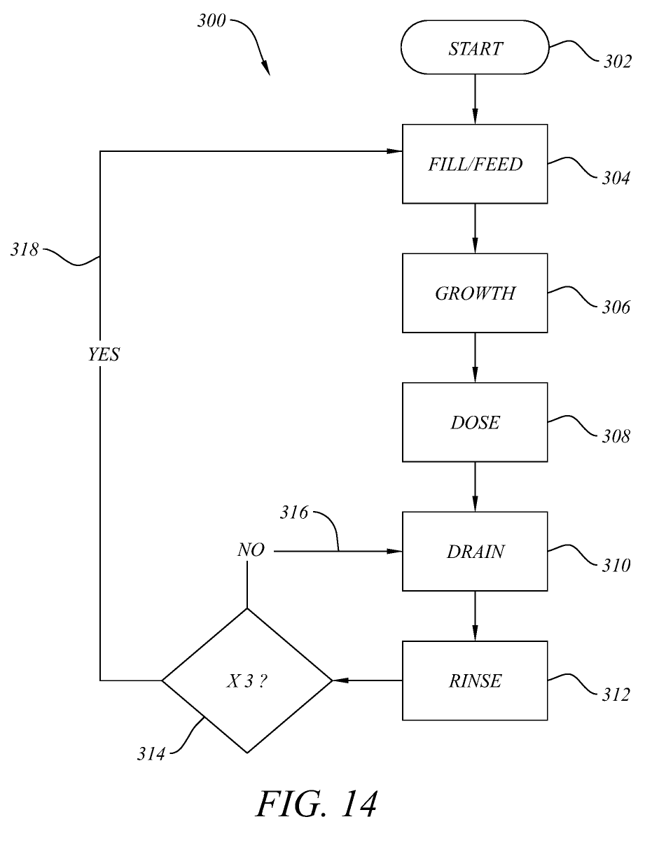

Referring to FIGS. 13-14, during startup of biomass generator 200 and initiation of Fill/Feed stage 304 of operating cycle 300 (FIG. 14), water circulation through bacteria growth chamber 202 is initially established by opening solenoid valve 224 to allow inlet water to flow into bacteria growth chamber 202 through flow lines 252, 254. Recirculation pump 208 is then activated to draw water through an outlet port in the bottom of bacteria growth chamber 202 through flow lines 258, 260 and heater 204, and return it to manifold 225 through recirculation return line 252. According to one embodiment of the invention, solenoid valve 224 allows about 2.5 liters of chlorinated inlet water into bacteria growth chamber 202, and then closes, after which recirculation pump 208 recirculates the water for about 3.5 minutes. If desired, solenoid pump 244 (FIG. 13) can be activated to draw disinfectant into manifold 225 from receptacle 242 through flow line 243 during this period to kill any undesirable bacteria that may be present in the wetted areas of biomass generator 200 or in the inlet water at startup. If needed, heater 204 disposed between bacteria growth chamber 202 and recirculation pump 208 can be activated to warm the recirculating water to a desired operating temperature that is beneficial to growing the bacteria. Referring to FIG. 13, when fluid circulation is established, solenoid valve 224 is again opened to allow about 0.5 liters of water to enter manifold 225. Solenoid pumps 236, 238, 240 are activated to pump desired quantities of liquid nutrient, bacteria spores and defoamer into the water stream through check valve 228. Solenoid valve 224 is then closed and recirculation pump 208 continues withdrawing the bacteria growth medium from the bottom of bacteria growth vessel and discharging it through manifold 225 back into bacteria growth chamber 202. At this point Growth stage 306 (FIG. 14) begins.

During Growth stage 306, air pump 218 and water heater 204 can be activated if needed. Temperature controller 206 is desirably provided to facilitate maintaining the desired temperature. After recirculating for the period needed for the bacteria to grow into a desired utility population, the process of using biomass generator 200 continues to Dose stage 308 (FIG. 14).

During Dose stage 308, peristaltic dosing pump 210 is desirably activated to draw recirculating bacteria growth medium from manifold 225 through inlet line 290 and to discharge the bacteria growth medium through dosing outlet port 212 (FIG. 13) at a controlled rate. During Dose stage 308, water heater 204 can be turned off if desired, but air pump 218 and recirculation pump 208 desirably continue to run. Once a desired amount of the bacteria growth medium has been discharged through dosing outlet port 212, peristaltic dosing pump 210 is turned off and any bacteria growth medium still being recirculated is drained from biomass generator by opening solenoid valve 246 to discharge any remaining liquid through drain lines 262, 264. This interval is depicted as Drain stage 310 in FIG. 14.

Following Drain stage 310, Rinse stage 312 is initiated by closing solenoid valve 246, opening solenoid valve 224 and allowing 5 liters of water to be introduced into bacteria growth chamber 202 through manifold 225 and flow line 254 while air pump 218 and recirculation pump 208 are still operating. As the rinse water is being introduced, solenoid pump 244 is again activated to pump liquid disinfectant, preferably bleach, into manifold 225 from receptacle 242 through flow line 243. Dosing pump 210 is also activated during Rinse stage 312, and circulation is continued until all liquid has been discharged. Referring to decision block 314 in FIG. 14, Rinse stage 312 is desirably continued until three rinse cycles are completed to ensure thorough cleanout and disinfecting prior to beginning another Fill/Feed stage 204 as indicated by arrow 318.

Referring again to FIGS. 8-9, 12, all the functional operations needed during the performance of operating cycle 300 of biomass generator 200 as described above are desirably performed by programmable electronic controller 284, which desirably comprises keypad 286 and a plurality of preprogrammed control options that are desirably implemented through use of an interactive digital display 288.

According to yet another embodiment of the invention, rather than providing an circulating a liquid disinfectant to disinfect the wetted surfaces, the apparatus is configured to automatically disinfect the wetted surfaces by heat sterilization of the recirculating bacteria growth medium prior to starting the bacteria growing cycle by heating and recirculating the liquid at a temperature of about 65° C. for one hour, and then allowing it to cool to about 35° C. before beginning timing of the bacteria growing cycle. Alternatively, the apparatus is configured to automatically heat recirculating purge water following harvesting of the bacteria growth medium to a temperature of about 65° C. for one hour, after which the recirculating water is discharged before refilling the bacteria growing chamber and introducing additional bacteria-containing liquid and liquid nutrient to commence another bacteria growth cycle. As used in this embodiment of the invention, “heat sterilize” means to raise the temperature of the recirculating liquid to a temperature and for a period of time that are sufficient to purge the system of harmful bacteria without substantially harming any desirable bacteria spores in the recirculating liquid. Heating the recirculating liquid to a temperature of about 65° C. for about one hour is believed to be sufficient for achieving this purpose. In this embodiment, an external heater can be provided through which the bacteria growth medium or water can be recirculated to reach and maintain the desired temperature. Where heated inlet water is recirculated following harvest of the bacteria growth medium, it may be desirably to fill the bacteria growth chamber slightly above the normal fill level to assure that all the wetted surfaces are heat sterilized. The following Example 2 further describes and explains an in situ biomass generator comprising a heat sterilization capability:

### Example 2

Tap water and about 10 mL Bacillus spore suspension, 60 mL nutrient broth and 5 mL defoamer are added per about 3 liters water to form a bacteria growth medium, which is then heated to raise the temperature of the medium to about 65° C. The heated growth medium is recirculated through the growth chamber and a heater (preferably part of the subject system but disposed external to the housing containing the bacteria growing chamber) for about one hour while maintaining the liquid temperature at about 65° C., after which the heater is turned off and the flow of recirculating growth medium is redirected to bypass the heater. Recirculation continues until the bacteria growth medium cools to a temperature of about 35° C., at which time an electronic timer initiates timing of a predetermined bacteria growth cycle. At the end of the bacteria growth cycle, the bacteria growth medium is harvested through the drain tube, and another cycle of operation can begin.

Alternatively, in circumstances where there is heavy contamination, dirty water, or the like, the subject apparatus can be selectively configured so that the bacteria growing chamber is overfilled with inlet water to a level above the normal fill level and the water is heated to a temperature of about 65° C. The water is then desirably recirculated between the growing chamber and a heater for one hour while maintaining the temperature at that level to heat sterilize the wetted surfaces and then discharged into a drain, after which new inlet water, liquid spore suspension and nutrient broth are loaded into the growing chamber to commence a new growing cycle.

Those of ordinary skill in the art will appreciate upon reading the present disclosure that other ancillary equipment such as water supply line connections, downstream flow lines, valves, intermediate storage tanks, and the like can also be used in combination with the present invention. Those of ordinary skill in the art will similarly appreciate upon reading this specification in conjunction with the accompanying drawing figures that other alterations and modifications to the subject system and apparatus can be made within the scope of the invention, and it is intended that the scope of the invention disclosed herein be limited only by the broadest interpretation of the appended claims to which the inventors are legally entitled.

We claim:

1. An in situ biomass generator comprising a housing, a bacteria growth chamber having at least one inlet port and at least one outlet port; an inlet manifold in which a bacteria growth medium comprising water, a suspension of starter bacteria and a liquid nutrient broth are pre-mixed and introduced into the growth chamber to initiate a bacteria growth cycle; a recirculation pump configured to receive bacteria growth medium from an outlet port of the growth chamber; a recirculation manifold in which the growth medium received from the growth chamber can be heated and aerated; a recirculation loop selectively returning the growth medium to the growth chamber; a valve that is selectively operable to close the recirculation loop and harvest bacteria growth medium by redirecting the outlet flow from the recirculation pump; a disinfectant that is automatically introduced into the inlet manifold and circulated to disinfect any wetted surface prior to initiating another bacteria growth cycle; and an electronic controller that controls the flow of liquids through the biomass generator.

2. The biomass generator of claim 1, further comprising a separate enclosure comprising a plurality of liquid containers, with at least one container each containing a suspension of starter bacteria, a nutrient broth, and a liquid disinfectant.

3. The biomass generator of claim 2 wherein the disinfectant is selected from the group consisting of bleach, hydrogen peroxide, and quaternary ammonium compounds.

4. The biomass generator of claim 1, further comprising a water inlet valve and a flow meter to control fluid flow into the bacteria growth chamber.

5. The biomass generator of claim 2 further comprising a plurality of peristaltic pumps that cooperate with the electronic controller to control the flows of starter bacteria, nutrient broth and disinfectant into the inlet manifold from the plurality of liquid containers.

6. The biomass generator of claim 1, further comprising a temperature sensor positioned to detect the temperature of the recirculating growth medium.

7. The biomass generator of claim 6, further comprising at least one resistance heating element configured to heat the recirculating growth medium.

8. The biomass generator of claim 1, further comprising a cover having a vent line.

9. The biomass generator of claim 1 wherein the electronic controller further comprises a timer.

10. The biomass generator of claim 9 wherein the electronic controller defines an operational cycle including a plurality of parameters and is configured to enable a user to selectively adjust the plurality of parameters.

11. The biomass generator of claim 10 wherein the plurality of parameters includes a liquid volume charged to the bacteria growth chamber, a liquid flow rate through each of the plurality of peristaltic pumps, a liquid recirculation rate to the bacteria growth chamber, a target temperature for the recirculating growth medium, the time interval during which the bacteria growth medium is recirculated, and the time interval during which the disinfectant is recirculated.

12. The biomass generator of claim 1, further comprising an air inlet line through which pressurized air is introduced into the liquid recirculation loop.

13. An in situ biomass generator system configured to produce bacteria-containing growth medium during a plurality of successive bacteria growing cycles, the system comprising in fluid communication a water inlet, a plurality of water flow lines, a bacteria growing chamber, a recirculating pump, and a heater, wherein the water inlet, recirculating pump and heater are configured and controlled by an electronic controller to disinfect the wetted surfaces by heat sterilization by receiving water from the water inlet into the bacteria growing chamber, heating the water to 65° C., and recirculating the water between the bacteria growing chamber and the heater for a sufficient period to disinfect the wetted surfaces.

14. A self-contained biomass generator configured for use in growing and discharging bacteria at or near an intended use site, the biomass generator comprising:
a housing;
a bacteria growth chamber;
water and air inlets;
receptacles and solenoid pumps for liquids containing bacteria or bacteria spores, nutrient, defoamer and disinfectant;
a recirculation pump;
an air pump;
a water heater;
a manifold;
a dosing pump; and
a programmable electronic controller.

15. The biomass generator of claim 14 wherein the dosing pump is configured to dispense controlled amounts of bacteria-containing growth medium for a desired end-use application.

16. The biomass generator of claim 14 wherein the programmable electronic controller is configured to operate the device through a plurality of bacteria growth cycles and to disinfect the wetted internal surfaces between growth cycles without requiring service or operator intervention.

17. The biomass generator of claim 14 configured so that the recirculating pump draws liquid from an outlet at or near the bottom of the bacteria growth chamber and returns it through a side wall of the bacteria growth chamber so as to cause continuous swirling movement of the liquid inside the bacteria growth chamber.

18. The biomass generator of claim 14 wherein the air pump discharges air into the manifold at a positive pressure.

19. The biomass generator of claim 18 wherein the air pump is configured to be controllable by the electronic controller to discharge air into the manifold at a desired pressure and flow rate.

20. The biomass generator of claim 14 wherein the water heater is disposed in fluid communication with and between an outlet from the bacteria growth chamber and an inlet into the recirculation pump.

21. The biomass generator of claim 14 wherein the housing has a lockable door.

22. The biomass generator of claim 14 wherein the receptacles are configured to receive and be used with polymeric bags containing each of the liquids containing bacteria or bacteria spores, nutrient, defoamer and disinfectant;

23. The biomass generator of claim 14 wherein the defoamer is white oil.

24. The biomass generator of claim 14 wherein the disinfectant is liquid bleach.

25. The biomass generator of claim 14 wherein the manifold comprises separate inlets for air, water, recirculated liquid, liquid nutrient, liquid containing bacteria or bacteria spores, liquid containing defoamer, and liquid bleach.

26. The biomass generator of claim 14 wherein the manifold comprises separate outlets for recirculated liquid, dosing, and a drain.

27. The biomass generator of claim 14 wherein the bacteria growth chamber further comprises a cover.

28. The biomass generator of claim 14 further comprising a drain solenoid valve configured to control fluid flow through a drain line disposed in fluid communication with liquid discharged by the recirculation pump.

29. The biomass generator of claim 28 wherein the bacteria growth chamber is vented into a drain line downstream of the drain solenoid valve.

30. The biomass generator of claim 28 wherein the bacteria growth chamber operates at atmospheric pressure.

31. A process for growing bacteria using the biomass generator of claim 14, comprising:
introducing water into the bacteria growth chamber;
introducing additive liquids containing bacteria or bacteria spores, nutrient and defoamer into the water to form a bacteria growth medium;
activating the recirculation pump to continuously circulate water and the additive liquids through the bacteria growth chamber and the recirculation pump;
growing the bacteria in bacteria growth medium while controlling the temperature and aeration of the bacteria growth medium; and
activating the dosing pump to dispense bacteria growth medium from the biomass generator at a controlled rate.

32. The process for growing bacteria of claim 31 further comprising dosing, wherein dosing is performed during about a two-hour period after a utility population of bacteria is present in the bacteria growth medium.

33. The process for growing bacteria of claim 31, further comprising disinfecting and rinsing, wherein disinfecting and rinsing are performed by introducing water and liquid disinfectant into the bacteria growth chamber after dosing is completed and thereafter recirculating the water and liquid disinfectant to disinfect surfaces of the biomass generator that are wetted by the recirculating water and liquid disinfectant.
